(19) United States
(12) Patent Application Publication
(10) Pub. No.: US 2005/0256146 A1
(43) Pub. Date: Nov. 17, 2005

(54) Alpha-7 nicotinic receptor agonists and stains in combination

(76) Inventor: Richard Keith, Wilmington, DE (US)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(21) Appl. No.: 10/525,783

(22) PCT Filed: Sep. 1, 2003
(86) PCT No.: PCT/SE03/01352

§ 371 Date: Feb. 28, 2005

(30) Foreign Application Priority Data
Sep. 2, 2002 (SE) 0202598-9

(51) Int. Cl. A61K 31/4747; A61K 31/401; A61K 31/366; A61K 31/225

(52) U.S. Cl. 514/278; 514/423; 514/460; 514/548

(57) ABSTRACT

Combinations of α7-nAChR agonists and statins, pharmaceutical compositions containing the same and methods of using the same useful for treatment or prophylaxis of neurological degenerative diseases.

### FIELD OF INVENTION

This invention is concerned with the treatment of neurological degenerative diseases and particularly with the treatment of Alzheimer's disease.

### BACKGROUND

The etiology of Alzheimer's disease is complex and not entirely understood. Current hypotheses point to the overproduction of the amyloid peptide Aβ as a causative factor in the cognitive deficits and neurodegeneration associated with Alzheimer's disease (Selkoe, 2001; Walsh et. al., 2002). In addition, epidemiological studies have shown that hypercholesterolemia is a risk factor for Alzheimer's disease (Jarvik et. al., 1995; Notkola et. al., 1998). Further, it has recently been shown that the administration of statins is associated with a decreased risk of Alzheimer's disease (Jick et. al., 2000; Wolozin et. al., 2000). Still further, another recent study has shown that the statin lovastatin reduced Aβ plasma levels in human subjects that had elevated plasma levels of low-density lipoprotein cholesterol (Buxbaum et. al., 2002).

Alpha-7 nicotinic receptors (α7-nAChR) are ligand-gated ion channels that allow for the entry into cells of calcium and other monovalent cations (Dani, 2001). α7-nAChR have been shown to play an important role in regulating neurotransmitter release, hippocampal synaptic function, neuroprotection against a variety of insults, and cognition (Dani, 2001; Dahas-Bailador et. al., 2000; Rezvani and Levin, 2001).

Recent studies imply an interaction between Aβ and α7-nAChR that may contribute to the pathophysiology of Alzheimer's disease. Aβ has been shown to potently inhibit α7-nAChR (Liu et. al., 2001). It has been proposed that this inhibitory effect of Aβ on α7-nAChR function may contribute to cognitive deficits in Alzheimer's disease. Neurodegeneration induced by the activation of NMDA glutamatergic receptors is also enhanced in the presence of Aβ (Kihara et. al., 2001). This Aβ induced neurodegeneration is inhibited by activation of α7-nAChR.

### BACKGROUND REFERENCES

Buxbaum J D, Cullen E I and Friedhoff L T: Pharmacological concentrations of the HMG-CoA reductase inhibitor lovastatin decrease the formation of the Alzheimer beta-amyloid peptide in vitro and in patients. Frontiers in Bioscience 7:a50-a59, 2002.

Dajas-Bailador F A, Lima P A and Wonnacott S: The α7 nicotinic receptor subtype mediates nicotine protection against NMDA excitotoxicity in primary hippocampal cultures through a Ca²⁺ dependent mechanism. Neuropharmacology 39:2799-2807, 2000.

Dani J A: Overview of nicotinic receptors and their roles in the central nervous system. Biol Psychiatry 49:166-174, 2001.

Kihara T, Shimohama S, Sawada H, Honda K, Nakamizo T, Shibasaki H, Toshiaki K, and Akaike A: α7 Nicotinic receptor transduces signals to phohphatidylinositol 3-kinase to block Aβ-amyloid-induced neurotoxicity. J Biol Chem 276:13541-13546, 1998.

Jick H, Zornberg G L, Jick S S, Seshadri S, and Drachman D A: Statins and the risk of dementia. Lancet 356:1627-1631, 2000.

Jarvik G P, Wijsman E M, Kukull W A, Schellenberg G D, Yu C and Larson E B: Interactions of apolipoprotein E genotype, total cholesterol, age, and sex in prediction of Alzheimer's disease. Neurology 45:1092-1096, 1995.

Liu Q, Kawai H and Berg D K: b-Amyloid peptide blocks the response of α7-containing nicotinic receptors on hippocampal neurons. Proc Natl Acad Sci 97:10197-10202, 2001.

Notkola I L, Sulkava R, Pekkanen J, Erkinjuntti T, Ehnholm C, Kivinen P, Tuomilehto J and Nissinen A: Serum total cholesterol, apolipoprotein E epsilon 4 allele, and Alzheimer's disease. Neuroepidememiology 17:14-20, 1998.

Rezvani A H and Levin E D: Cognitive effects of nicotine. Biol Psychiatry 49:258-267, 2001.

Selkoe, D J: Alzheimer's disease: Genes, proteins, and therapy. Physiological Reviews 81:741-766, 2001.

Walsh D M, Klyubin I, Fadeeva J V, Cullen W K, and Selkoe, D J: Naturally secreted oligomers of amyloid β protein potently inhibit hippocampal long-term potentiation in vivo. Nature 416:535-539, 2002.

Wolozin B, Kellman W, Ruosseau P, Celesia G G, and Siegel G: Decreased prevalence of Alzheimer's disease associated with 3-hydroxy-3-methyglutaryl coenzyme A reductase inhibitors. Arch Neruol 57:1439-1443, 2000.

### DESCRIPTION OF THE INVENTION

We have discovered that statins and α7-nAChR agonists in combination have the potential to alter the pathophysiology of Alzheimer's disease and symptoms. The different mechanisms by which statins and α7-nAChR agonists operate—statins by reducing the formation of the neurotoxic substance Aβ and α7-nAChR agonists by blocking the cognitive impairing and neurotoxic effects of Aβ—imply that a statin and an α7-nAChR in combination will synergistically benefit patients suffering with neurological degenerative diseases and particularly patients suffering with Alzheimer's disease.

In one aspect the invention is a method for treating neurological degenerative diseases and particularly Alzheimer's disease comprising treatment with a combination comprising an α7-nAChR agonist and a statin.

A combination suitable for practicing the invention comprises a statin selected from atorvastatin, cerivastatin, fluvastatin, lovastatin, pravastatin sodium, simvastatin or rosuvastatin, or a pharmaceutically-acceptable salt thereof and an α7-nAChR agonist selected from spiro[1-azabicyclo[2.2.2]octane-3,5′-oxazolidine]-2′-one,

(+)-spiro[1-azabicyclo[2.2.2]octane-3,5′-oxazolidine]-2′-one,

(−)-spiro[1-azabicyclo[2.2.2]octane-3,5′-oxazolidine]-2′-one,

spiro[1-azabicyclo[2.2.1]heptan-3,5′-oxazolidin-2′-one],

3′-methyl spiro-[1-azabicyclo[2.2.2]octane-3,5′-oxazolidin-2′-one],

spiro[1-azabicyclo[2.2.2]octane-3,2′-(3′H)-furo[2,3-b]pyridine],

5′-bromospiro[1-azabicyclo[2.2.2]octane-3,2′-(3′H)-furo[2,3-b]pyridine],

5′-phenylspiro[1-azabicyclo[2.2.2]octane-3,2′-(3′H)-furo[2,3-b]pyridine],

5′-nitrospiro[1-azabicyclo[2.2.2]octane-3,2′-(3′H)-furo[2,3-b]pyridine),

1′-chlorospiro[1-azabicyclo[2.2.2]octane-3,2′-(3′H)-furo[2,3-b]isoquinoline],

5′-(phenylcarboxamido)spiro[1-azabicyclo[2.2.2]octane-3,2′-(3′H)-furo[2,3-b]pyridine],

5′-(phenylaminocarbonylamino)spiro[1-azabicyclo[2.2.2]octane-3,2′-(3′H)-furo[2,3-b]pyridine],

5′-(phenylsulfonylamido)spiro[1-azabicyclo[2.2.2]octane-3,2′-(3′H)-furo[2,3-b]pyridine],

5′-aminospiro[1-azabicyclo[2.2.2]octane-3,2′-(3′H)-furo[2,3-b]pyridine],

5′-N-methylaminospiro[1-azabicyclo[2.2.2]octane-3,2′-(3′H)-furo[2,3-b]pyridine],

5′-N,N-dimethylaminospiro[1-azabicyclo[2.2.2]octane-3,2′-(3′H)-furo[2,3-b]pyridine],

5′-N,N-diethylaminospiro[1-azabicyclo[2.2.2]octane-3,2′-(3′H)-furo[2,3-b]pyridine],

5′-N-ethylaminospiro[1-azabicyclo[2.2.2]octane-3,2′-(3′H)-furo[2,3-b]pyridine],

5′-N-benzylaminospiro[1-azabicyclo[2.2.2]octane-3,2′-(3′H)-furo[2,3-b]pyridine],

5′-N-formamidospiro[1-azabicyclo[2.2.2]octane-3,2′-(3′H)-furo[2,3-b]pyridine],

5′-N-acetamidospiro[1-azabicyclo[2.2.2]octane-3,2′-(3′H)-furo[2,3-b]pyridine],

spiro[1-azabicyclo[2.2.2]octane-3,2′-(3′H)-furo[2,3-b]isoquinoline],

spiro[1-azabicyclo[2.2.2]octane-3,2′-(3′H)-furo[2,3-b]quinoline],

5′-ethenylspiro[1-azabicyclo[2.2.2]octane-3,2′-(3′H)-furo[2,3-b]pyridine],

5′-(E)-(phenylethenyl)spiro[1-azabicyclo[2.2.2]octane-3,2′-(3′H)-furo[2,3-b]pyridine],

5′-(4-morpholino)spiro[1-azabicyclo[2.2.2]octane-3,2′-(3′H)-furo[2,3-b]pyridine],

5′-(1-azetidinyl)spiro[1-azabicyclo[2.2.2]octane-3,2′-(3′H)-furo[2,3-b]pyridine],

5′-(E)-(2-(4-pyridyl)ethenyl)spiro[1-azabicyclo[2.2.2]octane-3,2′-(3′H)-furo[2,3-b]pyridine],

5′-(E)-(2-(2-pyridyl)ethenyl)spiro[1-azabicyclo[2.2.2]octane-3,2′-(3′H)-furo[2,3-b]pyridine],

5′-(2-trimethylsilylethynyl)spiro[1-azabicyclo[2.2.2]octane-3,2′-(3′H)-furo[2,3-b]pyridine],

5′-ethynylspiro[1-azabicyclo[2.2.2]octane-3,2′-(3′H)-furo[2,3-b]pyridine],

5′-(2-furyl)spiro[1-azabicyclo[2.2.2]octane-3,2′-(3′H)-furo[2,3-b]pyridine],

5′-(3-pyridyl)spiro[1-azabicyclo[2.2.2]octane-3,2′-(3′H)-furo[2,3-b]pyridine],

5′-methylspiro[1-azabicyclo[2.2.2]octane-3,2′-(3′H)-furo[2,3-b]pyridine],

spiro[1-azabicyclo[2.2.2]octane-3,2′-(3′H)-furo[2,3-b]pyridine-5′carbonitrile],

spiro[1-azabicyclo[2.2.2]octane-3,2′-(3′H)-furo[2,3-b]pyridine-5′carboxamide],

5′-N′-(3-chlorophenyl)aminocarbonylminospiro[1-azabicyclo[2.2.2]octane-3,2′-(3′H)-furo[2,3-b]pyridine],

5′-N′-(2-nitrophenyl)aminocarbonylaminospiro[1-azabicyclo[2.2.2]octane-3,2′-(3′H)-furo[2,3-b]pyridine],

4′-chlorospiro[1-azabicyclo[2.2.2]octane-3,2′-(3′H)-furo[2,3-b]pyridine],

4′-methoxyspiro[1-azabicyclo[2.2.2]octane-3,2′-(3′H)-furo[2,3-b]pyridine],

4′-phenylthiospiro[1-azabicyclo[2.2.2]octane-3,2′-(3′H)-furo[2,3-b]pyridine],

4′-(N-2-aminoethyl)aminospiro[1-azabicyclo[2.2.2]octane-3,2′-(3′H)-furo[2,3-b]pyridine],

4′-phenylaminospiro[1-azabicyclo[2.2.2]octane-3,2′-(3′H)-furo[2,3-b]pyridine],

4′-methylaminospiro[1-azabicyclo[2.2.2]octane-3,2′-(3′H)-furo[2,3-b]pyridine],

4′-(4-N-methylpiperazin-1-yl)spiro[1-azabicyclo[2.2.2]octane-3,2′-(3′H)-furo[2,3-b]pyridine],

4′-chloro-spiro[1-azabicyclo[2.2.2]octane-3,2′-(3′H)-furo[3,2-c]pyridine],

spiro[1-azabicyclo[2.2.2]octane-3,2′-(3′H)-furo[3,2-c]pyridine],

spiro[1-azabicyclo[2.2.2]octane-3,2′(3′H)-furo(2,3-b]pyridine-7′-oxide],

spiro[1-azabicyclo[2.2.2]octane-3,2′(3′H)-furo[2,3-b]pyridine-6′-carbonitrile],

6′-chlorospiro[1-azabicyclo[2.2.2]octane-3,2′(3′H)-furo[2,3-b]pyridine],

6′-fluorospiro[1-azabicyclo[2.2.2]octane-3,2′(3′H)-furo[2,3-b]pyridine],

N-(1-azabicyclo[2.2.2]oct-3-yl)(5-phenylfuran-2-carboxamide),

N-(1-azabicyclo[2.2.2]oct-3-yl)(5-(3-fluorophenyl)furan-2-carboxamide),

N-(1-azabicyclo[2.2.2]oct-3-yl)(3-(3-thienyl)benzamide),

N-(1-azabicyclo[2.2.2]oct-3-yl)(3-phenylbenzamide),

N-(1-azabicyclo[2.2.2]oct-3-yl)(3-(3-pyridyl)benzamide),

N-(1-azabicyclo[2.2.2]oct-3-yl)(5-phenylthiophene-2-carboxamide),

N-(1-azabicyclo[2.2.2]oct-3-yl)(3-(3-methoxyphenyl)benzamide),

N-(1-azabicyclo[2.2.2]oct-3-yl)(3-(2-methoxyphenyl)benzamide),

N-(1-azabicyclo[2.2.2]oct-3-yl)(3-(3-(N-acetylamino)phenyl)benzamide),

N-(1-azabicyclo[2.2.2]oct-3-yl)(3-(3-fluorophenyl)benzamide),

N-(1-azabicyclo[2.2.2]oct-3-yl)(3-(3-methylphenyl)benzamide),

N-(1-azabicyclo[2.2.2]oct-3-yl)(3-(2-thienyl)benzamide),

N-(1-azabicyclo[2.2.2]oct-3-yl)(3-(3,5-dichlorophenyl)benzamide),

N-(1-azabicyclo[2.2.2]oct-3-yl)(3-(2-naphthyl)benzamide),

N-(1-azabicyclo[2.2.2]oct-3-yl)(3-(4-fluorophenyl)benzamide),

N-(1-azabicyclo[2.2.2]oct-3-yl)(5-(3-pyridyl)furan-2-carboxamide),

N-(1-azabicyclo[2.2.2]oct-3-yl)(5-(3-thienyl)furan-2-carboxamide),

N-(1-azabicyclo[2.2.2]oct-3-yl)(5-(2-benzo[b]furanyl)furan-2-carboxamide),

N-(1-azabicyclo[2.2.2]oct-3-yl)(5-(4-pyridyl)furan-2-carboxamide),

N-(1-azabicyclo[2.2.2]oct-3-yl)(5-(2-thienyl)furan-2-carboxamide),

N-(1-azabicyclo[2.2.2]oct-3-yl)(5-(3-methoxyphenyl)furan-2-carboxamide),

N-(1-azabicyclo[2.2.2]oct-3-yl)(5-(2-methoxyphenyl)furan-2-carboxamide),

N-(1-azabicyclo[2.2.2]oct-3-yl)(5-(4-fluorophenyl )furan-2-carboxamide),

N-(1-azabicyclo[2.2.2]oct-3-yl)(5-(2-naphthyl)furan-2-carboxamide),

N-(1-azabicyclo[2.2.2]oct-3-yl)(5-(3-methylphenyl)furan-2-carboxamide),

N-(1-azabicyclo[2.2.2]oct-3-yl)(5-(3-furyl)furan-2-carboxamide),

N-(1-azabicyclo[2.2.2]oct-3-yl)(5-(2-furyl)furan-2-carboxamide),

N-(1-azabicyclo[2.2.2]oct-3-yl)(5-(2-pyridyl)furan-2-carboxamide),

N-(1-azabicyclo[2.2.2]oct-3-yl)(5-(4-pyridyl)thiophene-2-carboxamide);

N-(1-azabicyclo[2.2.2]oct-3-yl)(5-(3-pyridyl)thiophene-2-carboxamide),

N-(1-azabicyclo[2.2.2]oct-3-yl)(5-(2-pyridyl)thiophene-2-carboxamide),

N-(1-azabicyclo[2.2.2]oct-3-yl)(4-(2-pyridyl)thiophene-2-carboxamide),

N-(1-azabicyclo[2.2.2]oct-3-yl)(4-(4-pyridyl)thiophene-2-carboxamide),

N-(1-azabicyclo[2.2.2]oct-3-yl)(4-(3-pyridyl)thiophene-2-carboxamide),

N-(1-azabicyclo[2.2.2]oct-3-yl)(5-(3-(N-acetylamino)phenyl)furan-2-carboxamide),

N-(1-azabicyclo[2.2.2]oct-3-yl)(5-(3-nitrophenyl)furan-2-carboxamide),

N-(1-azabicyclo[2.2.2]oct-3-yl)(5-(3-trifluoromethylphenyl)furan-2-carboxamide),

N-(1-azabicyclo[2.2.2]oct-3-yl)(5-(3-chlorophenyl)furan-2-carboxamide),

N-(1-azabicyclo[2.2.2]oct-3-yl)(5-(3-(N-acetylamino)phenyl)thiophene-2-carboxamide),

N-(1-azabicyclo[2.2.2]oct-3-yl)(5-(3-fluorophenyl)thiophene-2-carboxamide),

N-(1-azabicyclo[2.2.2]oct-3-yl )(5-(3- methoxyphenyl)thiophene-2-carboxamide),

N-(1-azabicyclo[2.2.2]oct-3-yl)(5-(3-ethoxyphenyl)thiophene-2-carboxamide),

N-(1-azabicyclo[2.2.2]oct-3-yl)(5-(3,5-dimethylisoxazol-4-yl)furan-2-carboxamide),

N-(1-azabicyclo[2.2.2]oct-3-yl)(5-(3,5-dimethylisoxazol-4-yl)thiophene-2-carboxamide),

N-(1-azabicyclo[2.2.2]oct-3-yl)(5-(3-aminophenyl)thiophene-2-carboxamide),

N-(1-azabicyclo[2.2.2]oct-3-yl)(5-(3-pyridyl)thiophene-3-carboxamide),

N-(1-azabicyclo[2.2.2]oct-3-yl)(5-(4-chlorophenyl)furan-2-carboxamide),

N-(1-azabicyclo[2.2.2]oct-3-yl)(5-(3-pyridyl)thiazole-3-carboxamide),

N-(1-azabicyclo[2.2.2]oct-3-yl)(5-(4-pyridyl)thiazole-3-carboxamide),

N-(1-azabicyclo[2.2.2]oct-3-yl)(5-(3-(N,N-dimethylamino)phenyl)thiophene-2-carboxamide),

N-(1-azabicyclo[2.2.2]oct-3-yl)(5-(8-quinolinyl)thiophene-2-carboxamide),

N-(1-azabicyclo[2.2.2]oct-3-yl)(5-phenylthiophene-3-carboxamide),

N-(1-azabicyclo[2.2.2]oct-3-yl)(4-phenylthiophene-2-carboxamide),

N-(1-azabicyclo[2.2.2]oct-3-yl)(5-(3-cyanophenyl)thiophene-2-carboxamide),

N-(1-azabicyclo[2.2.2]oct-3-yl)(5-(3-(N-methylamino)phenyl)thiophene-2-carboxamide),

N-(1-azabicyclo[2.2.2]oct-3-yl)(5-(3-hydroxyphenyl)thiophene-2-carboxamide),

N-(1-azabicyclo[2.2.2]oct-3-yl)(5-(3-pyridylamino)thiophene-2-carboxamide),

N-(1-azabicyclo[2.2.2]oct-3-yl)(5-(3-chlorophenyl)thiophene-2-carboxamide),

N-(1-aza-bicyclo[2.2.2]oct-3-yl)(5-(3-(4-morpholinyl)phenyl)thiophene-2-carboxamide),

N-(1-azabicyclo[2.2.2]oct-3-yl)(5-(3-(aminomethyl)phenyl)thiophene-2-carboxamide),

N-(1-azabicyclo[2.2.2]oct-3-yl)(5-phenoxythiophene-2-carboxamide),

N-(1-azabicyclo[2.2.2]oct-3-yl)(5-(3-aminophenyl)furan-2-carboxamide),

N-(1-azabicyclo[2.2.2]oct-3-yl)(5-(3-(N,N-dimethylamino)phenyl)furan-2-carboxamide),

N-(1-azabicyclo[2.2.2]oct-3-yl)(5-(3-formylphenyl)thiophene-2-carboxamide); N-(1-azabicyclo[2.2.2]oct-3-yl)(5-(3-(hydroxymethyl)phenyl)thiophene-2-carboxamide),

(R)-N-(1-azabicyclo[2.2.2]oct-3-yl)(5-phenylfuran-2-carboxamide),

(R)-N-(1-azabicyclo[2.2.2]oct-3-yl)(5-(3-fluorophenyl)furan-2-carboxamide),

(R)-N-(1-azabicyclo[2.2.2]oct-3-yl)(3-(3-thienyl)benzamide),

(R)-N-(1-azabicyclo[2.2.2]oct-3-yl)(3-phenylbenzamide),

(R)-N-(1-azabicyclo[2.2.2]oct-3-yl)(3-(3-pyridyl)benzamide),

(R)-N-(1-azabicyclo[2.2.2]oct-3-yl)(5-phenylthiophene-2-carboxamide),

(R)-N-(1-azabicyclo[2.2.2]oct-3-yl)(3-(3-methoxyphenyl)benzamide),

(R)-N-(1-azabicyclo[2.2.2]oct-3-yl)(3-(2-methoxyphenyl)benzamide),

(R)-N-(1-azabicyclo[2.2.2]oct-3-yl)(3-(3-(N-acetylamino)phenyl)benzamide);

(R)-N-(1-azabicyclo[2.2.2]oct-3-yl)(3-(3-fluorophenyl)benzamide),

(R)-N-(1-azabicyclo[2.2.2]oct-3-yl)(3-(3-methylphenyl)benzamide),

(R)-N-(1-azabicyclo[2.2.2]oct-3-yl)(3-(2-thienyl)benzamide),

(R)-N-(1-azabicyclo[2.2.2]oct-3-yl)(3-(3,5-dichlorophenyl)benzamide),

(R)-N-(1-azabicyclo[2.2.2]oct-3-yl)(3-(2-naphthyl)benzamide),

(R)-N-(1-azabicyclo[2.2.2]oct-3-yl)(3-(4-fluorophenyl)benzamide),

(R)-N-(1-azabicyclo[2.2.2]oct-3-yl)(5-(3-pyridyl)furan-2-carboxamide),

(R)-N-(1-azabicyclo[2.2.2]oct-3-yl)(5-(3-thienyl)furan-2-carboxamide),

(R)-N-(1-azabicyclo[2.2.2]oct-3-yl)(5-(2-benzo[b]furanyl)furan-2-carboxamide),

(R)-N-(1-azabicyclo[2.2.2]oct-3-yl)(5-(4-pyridyl)furan-2-carboxamide),

(R)-N-(1-azabicyclo[2.2.2]oct-3-yl)(5-(2-thienyl)furan-2-carboxamide),

(R)-N-(1-azabicyclo[2.2.2]oct-3-yl)(5-(3-methoxyphenyl)furan-2-carboxamide),

(R)-N-(1-azabicyclo[2.2.2]oct-3-yl)(5-(2-methoxyphenyl)furan-2-carboxamide),

(R)-N-(1-azabicyclo[2.2.2]oct-3-yl)(5-(4-fluorophenyl)furan-2-carboxamide),

(R)-N-(1-azabicyclo[2.2.2]oct-3-yl)(5-(2-naphthyl)furan-2-carboxamide),

(R)-N-(1-azabicyclo[2.2.2]oct-3-yl)(5-(3-methylphenyl)furan-2-carboxamide),

(R)-N-(1-azabicyclo[2.2.2]oct-3-yl)(5-(3-furyl)furan-2-carboxamide),

(R)-N-(1-azabicyclo[2.2.2]oct-3-yl)(5-(2-furyl)furan-2-carboxamide),

(R)-N-(1-azabicyclo[2.2.2]oct-3-yl)(5-(2-pyridyl)furan-2-carboxamide),

(R)-N-(1-azabicyclo[2.2.2]oct-3-yl)(5-(4-pyridyl)thiophene-2-carboxamide),

(R)-N-(1-azabicyclo[2.2.2]oct-3-yl)(5-(3-pyridyl)thiophene-2-carboxamide),

(R)-N-(1-azabicyclo[2.2.2]oct-3-yl)(5-(2-pyridyl)thiophene-2-carboxamide),

(R)-N-(1-azabicyclo[2.2.2]oct-3-yl)(4-(2-pyridyl)thiophene-2-carboxamide),

(R)-N-(1-azabicyclo[2.2.2]oct-3-yl)(4-(4-pyridyl)thiophene-2-carboxamide),

(R)-N-(1-azabicyclo[2.2.2]oct-3-yl)(4-(3-pyridyl)thiophene-2-carboxamide),

(R)-N-(1-azabicyclo[2.2.2]oct-3-yl)(5-(3-(N-acetylamino)phenyl)furan-2-carboxamide),

(R)-N-(1-azabicyclo[2.2.2]oct-3-yl)(5-(3-nitrophenyl)furan-2-carboxamide),

(R)-N-(1-azabicyclo[2.2.2]oct-3-yl)(5-(3-trifluoromethylphenyl)furan-2-carboxamide),

(R)-N-(1-azabicyclo[2.2.2]oct-3-yl)(5-(3-chlorophenyl)furan-2-carboxamide),

(R)-N-(1-azabicyclo[2.2.2]oct-3-yl)(5-(3-(N-acetylamino)phenyl)thiophene-2-carboxamide),

(R)-N-(1-azabicyclo[2.2.2]oct-3-yl)(5-(3-fluorophenyl)thiophene-2-carboxamide),

(R)-N-(1-azabicyclo[2.2.2]oct-3-yl )(5-(3-methoxyphenyl)thiophene-2-carboxamide),

(R)-N-(1-azabicyclo[2.2.2]oct-3-yl)(5-(3-ethoxyphenyl)thiophene-2-carboxamide),

(R)-N-(1-azabicyclo[2.2.2]oct-3-yl)(5-(3,5-dimethylisoxazol-4-yl)furan-2-carboxamide),

(R)-N-(1-azabicyclo[2.2.2]oct-3-yl)(5-(3,5-dimethylisoxazol-4-yl)thiophene-2-carboxamide),

(R)-N-(1-azabicyclo[2.2.2]oct-3-yl)(5-(3-aminophenyl)thiophene-2-carboxamide),

(R)-N-(1-azabicyclo[2.2.2]oct-3-yl)(5-(3-pyridyl)thiophene-3-carboxamide),

(R)-N-(1-azabicyclo[2.2.2]oct-3-yl)[5-(4-chlorophenyl)furan-2-carboxamide),

(R)-N-(1-azabicyclo[2.2.2]oct-3-yl)(5-(3-pyridyl)thiazole-3-carboxamide),

(R)-N-(1-azabicyclo[2.2.2]oct-3-yl)(5-(4-pyridyl)thiazole-3-carboxamide),

(R)-N-(1-azabicyclo[2.2.2]oct-3-yl)(5-(3-(N,N-dimethylamino)phenyl)thiophene-2-carboxamide),

(R)-N-(1-azabicyclo[2.2.2]oct-3-yl)(5-(8-quinolinyl)thiophene-2-carboxamide),

(S)-N-(1-azabicyclo[2.2.2]oct-3-yl)(5-(3-pyridyl)thiophene-2-carboxamide);

(S)-N-(1-azabicyclo[2.2.2]oct-3-yl)(5-(4-pyridyl)thiophene-2-carboxamide),

(S)-N-(1-azabicyclo[2.2.2]oct-3-yl)(5-(2-pyridyl)thiophene-2-carboxamide),

(S)-N-(1-azabicyclo[2.2.2]oct-3-yl)(5-phenylthiophene-2-carboxamide),

(R)-N-(1-azabicyclo[2.2.2]oct-3-yl)(5-phenylthiophene-3-carboxamide),

(R)-N-(1-azabicyclo[2.2.2]oct-3-yl)(4-phenylthiophene-2-carboxamide),

(R)-N-(1-azabicyclo[2.2.2]oct-3-yl)(5-(3-cyanophenyl)thiophene-2-carboxamide),

(R)-N-(1-azabicyclo[2.2.2]oct-3-yl)(5-(3-(N-methylamino)phenyl)thiophene-2-carboxamide),

(R)-N-(1-aza-bicyclo[2.2.2]oct-3-yl)(5-(3-hydroxyphenyl)thiophene-2-carboxamide),

(R)-N-(1-azabicyclo[2.2.2]oct-3-yl )(5-(3-pyridylamino)thiophene-2-carboxamide),

(R)-N-(1-azabicyclo[2.2.2]oct-3-yl)(5-(3-chlorophenyl)thiophene-2-carboxamide),

(R)-N-(1-azabicyclo[2.2.2]oct-3-yl )(5-(3-(4-morpholinyl)phenyl)thiophene-2-carboxamide),

(R)-N-(1-azabicyclo[2.2.2]oct-3-yl)(5-(3-(aminomethyl)phenyl)thiophene-2-carboxamide),

(R)-N-(1-azabicyclo[2.2.2]oct-3-yl )(5-phenoxythiophene-2-carboxamide),

(R)-N-(1-azabicyclo[2.2.2]oct-3-yl)(5-(3-aminophenyl)furan-2-carboxamide),

(R)-N-(1-azabicyclo[2.2.2]oct-3-yl)(5-(3-(N,N-dimethylamino)phenyl)furan-2-carboxamide),

(R)-N-(1-azabicyclo[2.2.2]oct-3-yl)(5-(3-formylphenyl)thiophene-2-carboxamide), or

(R)-N-(1-azabicyclo[2.2.2]oct-3-yl)(5-(3-(hydroxymethyl)phenyl)thiophene-2-carboxamide), or a pharmaceutically-acceptable salt thereof.

In general, it is contemplated that any statin when used in combination with any alpha-7-nAChR agonist will be useful in practicing the present invention.

Alpha-7-nAChR agonists contemplated to be useful in the present invention are described in international publications WO9606098, WO9730998. WO 9903859, WO9956745, WO0042044, WO0129034, WO0160821, WO0132622, WO0136417, WO0132619, WO0132620, WO0136417, WO0244176, WO0220521, WO0216358, WO0216357, WO0216356, WO0216355, WO0215662 and WO0217358 and in publications EP1219622, EP1184383, EP1184384, EP1184385, JP200203084. Statins contemplated to be useful in the present inventions are atorvastatin calcium (Lipitor), cerivastatin sodium (Baycol), fluvastatin sodium (Lescol), lovastatin (Mevacor), pravastatin sodium (Pravachol), simvastatin (Zocor) and rosuvastatin (Crestor).

In another aspect the invention is a pharmaceutical composition comprising a combination of an α7-nAChR agonist and a statin as described herein together with a pharmaceutically-acceptable diluent or excipient.

In another aspect the present invention comprises providing neuroprotection or analgesia in a method of treatment or prophylaxis of a condition or disorder involving reduced cholinergic function selected from Alzheimer's disease, cognitive or attention disorders, anxiety, depression, smoking cessation, schizophrenia, Tourette's syndrome, and Parkinson's disease which method comprises administering a therapeutically effective amount of a combination as defined in claim I to a patient.

In a particular aspect the method of the invention is a method for the treatment or prophylaxis of Alzheimer's disease.

A further aspect of the invention is the use of a combination of an α7-nAChR agonist and a statin as described herein in the preparation of a medicament for providing neuroprotection or analgesia in the treatment of a condition or disorder involving reduced cholinergic function selected from Alzheimer's disease, cognitive or attention disorders, anxiety, depression, smoking cessation, schizophrenia, Tourette's syndrome, and Parkinson's disease.

In a particular aspect the use of a combination of an α7-nAChR agonist and a statin as described herein is in the preparation of a medicament for the treatment or prophylaxis of Alzheimer's disease.

A particular combination for use in the present invention comprises rosuvastatin or a pharmaceutically-acceptable salt thereof and an α7-nAChR agonist selected from spiro[1-azabicyclo[2.2.2]octane-3,5′-oxazolidine]-2′-one, N-(1-azabicyclo[2.2.2]oct-3-yl)[E-3-(2-thienyl)propenamide], or (2′R)-5′-(3-furanyl)spiro[1-azabicyclo[2.2.2]octane-3,2′-(3′H)-furo[2,3-b]pyridine] or a pharmaceutically-acceptable salt thereof.

A particular pharmaceutical composition for use in the present invention comprises rosuvastatin or a pharmaceutically-acceptable salt thereof and an α7-nAChR agonist selected from spiro[1-azabicyclo[2.2.2]octane-3,5′-oxazolidine]-2′-one, N-(1-azabicyclo[2.2.2]oct-3-yl)[E-3-(2-thienyl)propenamide], or (2′R)-5′-(3-furanyl)spiro[1-azabicyclo[2.2.2]octane-3,2′-(3′H)-furo[2,3-b]pyridine] or a pharmaceutically-acceptable salt thereof together with a pharmaceutically acceptable diluent or carrier.

A particular method of the present invention is the provision of neuroprotection or analgesia for the treatment or prophylaxis of a condition or disorder involving reduced cholinergic function selected from Alzheimer's disease, cognitive or attention disorders, anxiety, depression, smoking cessation, schizophrenia, Tourette's syndrome, and Parkinson's disease which method comprises administering a therapeutically effective amount of a combination of rosuvastatin or a pharmaceutically-acceptable salt thereof and an α7-nAChR agonist selected from spiro[1-azabicyclo[2.2.2]octane-3,5′-oxazolidine]-2′-one, N-(1-azabicyclo[2.2.2]oct-3-yl)[E-3-(2-thienyl)propenamide], or (2′R)-5′-(3-furanyl)spiro[1-azabicyclo[2.2.2]octane-3,2′-(3′H)-furo[2,3-b]pyridine] or a pharmaceutically-acceptable salt thereof to a patient. In particular the method is useful for the treatment or prophylaxis Alzheimer's disease.

A particular embodiment of the invention is the use of a combination rosuvastatin or a pharmaceutically-acceptable salt thereof and an α7-nAChR agonist selected from spiro[1-azabicyclo[2.2.2]octane-3,5′-oxazolidine]-2′-one, N-(1-azabicyclo[2.2.2]oct-3-yl)[E-3-(2-thienyl)propenamide], or (2′R)-5′-(3-furanyl)spiro[1-azabicyclo[2.2.2]octane-3,2′-(3′H)-furo[2,3-b]pyridine] or a pharmaceutically-acceptable salt thereof in the preparation of a medicament providing neuroprotection or analgesia for the treatment of a condition or disorder involving reduced cholinergic function selected from Alzheimer's disease, cognitive or attention disorders, anxiety, depression, smoking cessation, schizophrenia, Tourette's syndrome, and Parkinson's disease. In particular the invention comprises the use of such a combination in the preparation of a medicament for the treatment of Alzheimer's disease.

Statins are compounds that inhibit HMG-CoA reductase, a rate-limiting enzyme in the biosynthetic pathway to cholesterol. Statins are conventionally used to reduce plasma levels of cholesterol in patients with cardiovascular disease but can also reduce Aβ serum levels in patients. Alpha-7-nAChR agonists beneficially activate α7-nACh receptors and are useful for treating cognitive deficits and in the treatment of a range of disorders involving reduced cholinergic function such as Alzheimer's disease, cognitive or attention disorders, anxiety, depression, smoking cessation, neuroprotection, schizophrenia, analgesia, Tourette's syndrome, and Parkinson's disease. Accordingly, the hypothetical basis of the present invention lies in the realization that statins, by reducing the formation of Aβ, may be particularly effective in combination with α7-nAChR agonists, which ameliorate cognitive deficits and inhibit neurodegeneration induced by Aβ, in the treatment of Alzheimer's disease. Therefore, the treatment of Alzheimer's disease with a combination of a statin and an α7-nAChR agonist will result in enhanced efficacy over either type of agent if administered alone.

Experimental

Assessment of the efficacy of a statin and an α7-nAChR agonist in combination in animal models is not straightforward. Existing experimental models of Alzhiemer's disease include transgenic mice, which over express Aβ, and animals with surgically generated fimbria-fornix lesions. These models and the uses to which they may be put are known, understood and appreciated by those of skill in the relevant art. Transgenic mice which over express Aβ exhibit some of the clinical manifestations of Alzheimer's disease, e.g., plaque deposition and, in some cases, cognitive deficits, but neurodegeneration is not observed. Animals with fimbria-fornix lesions have cognitive and learning deficits and have been used to assess potential approaches to treat neurodegeneration. No single experimental model exhibits the entire pathophysiological complex of Alzheimer's disease. However, to the extent that these models do mimic the pathophysiology of Alzheimer's disease they may be used to assess the effect of a statin and an α7-nAChR agonist in combination.

1.-12. (canceled)

13. A combination comprising:
a statin selected from atorvastatin, cerivastatin, fluvastatin, lovastatin, pravastatin sodium, simvastatin or rosuvastatin, or a pharmaceutically-acceptable salt thereof and
an α7-nAChR agonist.

14. A combination according to claim 13 wherein said α7-nAChR agonist is selected from spiro[1-azabicyclo[2.2.2]octane-3,5′-oxazolidine]-2′-one,
(+)-spiro[1-azabicyclo[2.2.2]octane-3,5′-oxazolidine]-2′-one,
(−)-spiro[1-azabicyclo[2.2.2]octane-3,5′-oxazolidine]-2′-one,
spiro[1-azabicyclo[2.2.1]heptan-3,5′-oxazolidin-2′-one],
3′-methyl spiro-[1-azabicyclo[2.2.2]octane-3,5′-oxazolidin-2′-one],
spiro[1-azabicyclo[2.2.2]octane-3,2′-(3′H)-furo[2,3-b]pyridine],
5′-bromospiro[1-azabicyclo[2.2.2]octane-3,2′-(3′H)-furo[2,3-b]pyridine],
5′-phenylspiro[1-azabicyclo[2.2.2]octane-3,2′-(3′H)-furo[2,3-b]pyridine],
5′-nitrospiro[1-azabicyclo[2.2.2]octane-3,2′-(3′H)-furo[2,3-b]pyridine],
1′-chlorospiro[1-azabicyclo[2.2.2]octane-3,2′-(3′H)-furo[2,3-b]isoquinoline],
5′-(phenylcarboxamido)spiro[1-azabicyclo[2.2.2]octane-3,2′-(3′H)-furo[2,3-b]pyridine],
5′-(phenylaminocarbonylamino)spiro[1-azabicyclo[2.2.2]octane-3,2′-(3′H)-furo[2,3-b]pyridine],
5′-(phenylsulfonylamido)spiro[1-azabicyclo[2.2.2]octane-3,2′-(3′H)-furo[2,3-b]pyridine],
5′-aminospiro[1-azabicyclo[2.2.2]octane-3,2′-(3′H)-furo[2,3-b]pyridine],
5′-N-methylaminospiro[1-azabicyclo[2.2.2]octane-3,2′-(3′H)-furo[2,3-b]pyridine],
5′-N,N-dimethylaminospiro[1-azabicyclo[2.2.2]octane-3,2′-(3′H)-furo[2,3-b]pyridine],
5′-N,N-diethylaminospiro[1-azabicyclo[2.2.2]octane-3,2′-(3′H)-furo[2,3-b]pyridine],
5′-N-ethylaminospiro[1-azabicyclo[2.2.2]octane-3,2′-(3′H)-furo[2,3-b]pyridine],
5′-N-benzylaminospiro[1-azabicyclo[2.2.2]octane-3,2′-(3′H)-furo [2,3-b]pyridine],
5′-N-formamidospiro[1-azabicyclo[2.2.2]octane-3,2′-(3′H)-furo[2,3-b]pyridine],
5′-N-acetamidospiro[1-azabicyclo[2.2.2]octane-3,2′-(3′H)-furo[2,3-b]pyridine],
spiro[1-azabicyclo[2.2.2]octane-3,2′-(3′H)-furo[2,3-b]isoquinoline],
spiro[1-azabicyclo[2.2.2]octane-3,2′-(3′H)-furo[2,3-b]quinoline],
5′-ethenylspiro[1-azabicyclo[2.2.2]octane-3,2′-(3′H)-furo[2,3-b]pyridine],
5′-(E)-(phenylethenyl)spiro[1-azabicyclo[2.2.2]octane-3,2′-(3′H)-furo[2,3-b]pyridine],
5′-(4-morpholino)spiro[1-azabicyclo[2.2.2]octane-3,2′-(3′H)-furo[2,3-b]pyridine],
5′-(1-azetidinyl)spiro[1-azabicyclo[2.2.2]octane-3,2′-(3′H)-furo[2,3-b]pyridine],
5′-(E)-(2-(4-pyridyl)ethenyl)spiro[1-azabicyclo[2.2.2]octane-3,2′-(3′H)-furo[2,3-b]pyridine],
5′-(E)-(2-(2-pyridyl)ethenyl)spiro[1-azabicyclo [2.2.2]octane-3,2′-(3′H)-furo[2,3-b]pyridine],
5′-(2-trimethylsilylethynyl)spiro[1-azabicyclo[2.2.2]octane-3,2′-(3′H)-furo[2,3-b]pyridine],
5′-ethynylspiro[1-azabicyclo[2.2.2]octane-3,2′-(3′H)-furo[2,3-b]pyridine],
5′-(2-furyl)spiro[1-azabicyclo[2.2.2]octane-3,2′-(3′H)-furo[2,3-b]pyridine],
5′-(3-pyridyl)spiro[1-azabicyclo[2.2.2]octane-3,2′-(3′H)-furo[2,3-b]pyridine],
5′-methylspiro[1-azabicyclo[2.2.2]octane-3,2′-(3′H)-furo [2,3-b]pyridine],
spiro[1-azabicyclo[2.2.2]octane-3,2′-(3′H)-furo[2,3-b]pyridine-5′carbonitrile],
spiro[1-azabicyclo[2.2.2]octane-3,2′-(3′H)-furo[2,3-b]pyridine-5′carboxamide],
5′-N′-(3-chlorophenyl)aminocarbonylminospiro[1-azabicyclo[2.2.2]octane-3,2′-(3′H)-furo[2,3-b]pyridine],
5′-N′-(2-nitrophenyl)aminocarbonylaminospiro[1-azabicyclo[2.2.2]octane-3,2′-(3′H)-furo[2,3-b]pyridine],
4′-chlorospiro[1-azabicyclo[2.2.2]octane-3,2′-(3′H)-furo[2,3-b]pyridine],
4′-methoxyspiro[1-azabicyclo[2.2.2]octane-3,2′-(3′H)-furo [2,3-b]pyridine],
4′-phenylthiospiro[1-azabicyclo[2.2.2]octane-3,2′-(3′H)-furo[2,3-b]pyridine],
4′-(N-2-aminoethyl)aminospiro[1-azabicyclo[2.2.2]octane-3,2′-(3′H)-furo[2,3-b]pyridine],
4′-phenylaminospiro[1-azabicyclo[2.2.2]octane-3,2′-(3′H)-furo[2,3-b]pyridine],
4′-methylaminospiro[1-azabicyclo[2.2.2]octane-3,2′-(3′H)-furo[2,3-b]pyridine],
4′-(4-N-methylpiperazin-1-yl)spiro[1-azabicyclo[2.2.2]octane-3,2′-(3′H)-furo[2,3-b]pyridine],
4′-chloro-spiro[1-azabicyclo[2.2.2]octane-3,2′-(3′H)-furo[3,2-c]pyridine],
spiro[1-azabicyclo[2.2.2]octane-3,2′-(3′H)-furo[3,2-c]pyridine],
spiro[1-azabicyclo[2.2.2]octane-3,2′(3′H)-furo[2,3-b]pyridine-7′-oxide],
spiro[1-azabicyclo[2.2.2]octane-3,2′(3′H)-furo[2,3-b]pyridine-6′-carbonitrile],
6′-chlorospiro[1-azabicyclo[2.2.2]octane-3,2′(3′H)-furo[2,3-b]pyridine],
6′-fluorospiro[1-azabicyclo[2.2.2]octane-3,2′(3′H)-furo[2,3-b]pyridine],
N-(1-azabicyclo[2.2.2]oct-3-yl)(5-phenylfuran-2-carboxamide),
N-(1-azabicyclo[2.2.2]oct-3-yl)(5-(3-fluorophenyl)furan-2-carboxamide),
N-(1-azabicyclo[2.2.2]oct-3-yl)(3-(3-thienyl)benzamide),
N-(1-azabicyclo[2.2.2]oct-3-yl)(3-phenylbenzamide),
N-(1-azabicyclo[2.2.2]oct-3-yl)(3-(3-pyridyl)benzamide),
N-(1-azabicyclo[2.2.2]oct-3-yl)(5-phenylthiophene-2-carboxamide),
N-(1-azabicyclo[2.2.2]oct-3-yl)(3-(3-methoxyphenyl)benzamide),
N-(1-azabicyclo[2.2.2]oct-3-yl)(3-(2-methoxyphenyl)benzamide),
N-(1-azabicyclo[2.2.2]oct-3-yl)(3-(3-(N-acetylamino)phenyl)benzamide),
N-(1-azabicyclo[2.2.2]oct-3-yl)(3-(3-fluorophenyl)benzamide),
N-(1-azabicyclo[2.2.2]oct-3-yl)(3-(3-methylphenyl)benzamide),
N-(1-azabicyclo[2.2.2]oct-3-yl)(3-(2-thienyl)benzamide),
N-(1-azabicyclo[2.2.2]oct-3-yl)(3-(3,5-dichlorophenyl)benzamide),
N-(1-azabicyclo[2.2.2]oct-3-yl)(3-(2-naphthyl)benzamide),
N-(1-azabicyclo[2.2.2]oct-3-yl)(3-(4-fluorophenyl)benzamide),
N-(1-azabicyclo[2.2.2]oct-3-yl)(5-(3-pyridyl)furan-2-carboxamide),
N-(1-azabicyclo[2.2.2]oct-3-yl)(5-(3-thienyl)furan-2-carboxamide),
N-(1-azabicyclo[2.2.2]oct-3-yl)(5-(2-benzo[b]furanyl)furan-2-carboxamide),
N-(1-azabicyclo[2.2.2]oct-3-yl)(5-(4-pyridyl)furan-2-carboxamide),
N-(1-azabicyclo[2.2.2]oct-3-yl)(5-(2-thienyl)furan-2-carboxamide),
N-(1-azabicyclo[2.2.2]oct-3-yl)(5-(3-methoxyphenyl)furan-2-carboxamide),
N-(1-azabicyclo[2.2.2]oct-3-yl)(5-(2-methoxyphenyl)furan-2-carboxamide),
N-(1-azabicyclo[2.2.2]oct-3-yl)(5-(4-fluorophenyl)furan-2-carboxamide),
N-(1-azabicyclo[2.2.2]oct-3-yl)(5-(2-naphthyl)furan-2-carboxamide),
N-(1-azabicyclo[2.2.2]oct-3-yl)(5-(3-methylphenyl)furan-2-carboxamide),
N-(1-azabicyclo[2.2.2]oct-3-yl)(5-(3-furyl)furan-2-carboxamide),
N-(1-azabicyclo[2.2.2]oct-3-yl)(5-(2-furyl)furan-2-carboxamide),
N-(1-azabicyclo[2.2.2]oct-3-yl)(5-(2-pyridyl)furan-2-carboxamide),
N-(1-azabicyclo[2.2.2]oct-3-yl)(5-(4-pyridyl)thiophene-2-carboxamide),
N-(1-azabicyclo[2.2.2]oct-3-yl)(5-(3-pyridyl)thiophene-2-carboxamide),
N-(1-azabicyclo[2.2.2]oct-3-yl)(5-(2-pyridyl)thiophene-2-carboxamide),
N-(1-azabicyclo[2.2.2]oct-3-yl)(4-(2-pyridyl)thiophene-2-carboxamide),
N-(1-azabicyclo[2.2.2]oct-3-yl)(4-(4-pyridyl)thiophene-2-carboxamide),
N-(1-azabicyclo[2.2.2]oct-3-yl)(4-(3-pyridyl)thiophene-2-carboxamide),
N-(1-azabicyclo[2.2.2]oct-3-yl)(5-(3-(N-acetylamino)phenyl)furan-2-carboxamide),
N-(1-azabicyclo[2.2.2]oct-3-yl)(5-(3-nitrophenyl)furan-2-carboxamide),
N-(1-azabicyclo[2.2.2]oct-3-yl)(5-(3-trifluoromethylphenyl)furan-2-carboxamide),
N-(1-azabicyclo[2.2.2]oct-3-yl)(5-(3-chlorophenyl)furan-2-carboxamide),
N-(1-azabicyclo[2.2.2]oct-3-yl)(5-(3-(N-acetylamino)phenyl)thiophene-2-carboxamide),
N-(1-azabicyclo[2.2.2]oct-3-yl)(5-(3-fluorophenyl)thiophene-2-carboxamide),
N-(1-azabicyclo[2.2.2]oct-3-yl)(5-(3-methoxyphenyl)thiophene-2-carboxamide),
N-(1-azabicyclo[2.2.2]oct-3-yl)(5-(3-ethoxyphenyl)thiophene-2-carboxamide),
N-(1-azabicyclo[2.2.2]oct-3-yl)(5-(3,5-dimethylisoxazol-4-yl)furan-2-carboxamide),
N-(1-azabicyclo[2.2.2]oct-3-yl)(5-(3,5-dimethylisoxazol-4-yl)thiophene-2-carboxamide),
N-(1-azabicyclo[2.2.2]oct-3-yl)(5-(3-aminophenyl)thiophene-2-carboxamide),
N-(1-azabicyclo[2.2.2]oct-3-yl)(5-(3-pyridyl)thiophene-3-carboxamide),
N-(1-azabicyclo[2.2.2]oct-3-yl)(5-(4-chlorophenyl)furan-2-carboxamide),
N-(1-azabicyclo[2.2.2]oct-3-yl)(5-(3-pyridyl)thiazole-3-carboxamide),
N-(1-azabicyclo[2.2.2]oct-3-yl)(5-(4-pyridyl)thiazole-3-carboxamide),
N-(1-azabicyclo[2.2.2]oct-3-yl)(5-(3-(N,N-dimethylamino)phenyl)thiophene-2-carboxamide),
N-(1-azabicyclo[2.2.2]oct-3-yl)(5-(8-quinolinyl)thiophene-2-carboxamide),
N-(1-azabicyclo[2.2.2]oct-3-yl)(5-phenylthiophene-3-carboxamide),
N-(1-azabicyclo[2.2.2]oct-3-yl)(4-phenylthiophene-2-carboxamide),
N-(1-azabicyclo[2.2.2]oct-3-yl)(5-(3-cyanophenyl)thiophene-2-carboxamide),
N-(1-azabicyclo[2.2.2]oct-3-yl)(5-(3-(N-methylamino)phenyl)thiophene-2-carboxamide),
N-(1-azabicyclo[2.2.2]oct-3-yl)(5-(3-hydroxyphenyl)thiophene-2-carboxamide),
N-(1-azabicyclo[2.2.2]oct-3-yl)(5-(3-pyridylamino)thiophene-2-carboxamide),
N-(1-azabicyclo[2.2.2]oct-3-yl)(5-(3-chlorophenyl)thiophene-2-carboxamide),
N-(1-aza-bicyclo[2.2.2]oct-3-yl)(5-(3-(4-morpholinyl)phenyl)thiophene-2-carboxamide),
N-(1-azabicyclo[2.2.2]oct-3-yl)(5-(3-(aminomethyl)phenyl)thiophene-2-carboxamide),
N-(1-azabicyclo[2.2.2]oct-3-yl)(5-phenoxythiophene-2-carboxamide),
N-(1-azabicyclo[2.2.2]oct-3-yl)(5-(3-aminophenyl)furan-2-carboxamide),
N-(1-azabicyclo[2.2.2]oct-3-yl)(5-(3-(N,N-dimethylamino)phenyl)furan-2-carboxamide),
N-(1-azabicyclo[2.2.2]oct-3-yl)(5-(3-formylphenyl)thiophene-2-carboxamide); N-(1-azabicyclo[2.2.2]oct-3-yl)(5-(3-(hydroxymethyl)phenyl)thiophene-2-carboxamide),
(R)-N-(1-azabicyclo[2.2.2]oct-3-yl)(5-phenylfuran-2-carboxamide),
(R)-N-(1-azabicyclo[2.2.2]oct-3-yl)(5-(3-fluorophenyl)furan-2-carboxamide),
(R)-N-(1-azabicyclo[2.2.2]oct-3-yl)(3-(3-thienyl)benzamide),
(R)-N-(1-azabicyclo[2.2.2]oct-3-yl)(3-phenylbenzamide),
(R)-N-(1-azabicyclo[2.2.2]oct-3-yl)(3-(3-pyridyl)benzamide),
(R)-N-(1-azabicyclo[2.2.2]oct-3-yl)(5-phenylthiophene-2-carboxamide),
(R)-N-(1-azabicyclo[2.2.2]oct-3-yl)(3-(3-methoxyphenyl)benzamide),
(R)-N-(1-azabicyclo[2.2.2]oct-3-yl)(3-(2-methoxyphenyl)benzamide),
(R)-N-(1-azabicyclo[2.2.2]oct-3-yl)(3-(3-(N-acetylamino)phenyl)benzamide);
(R)-N-(1-azabicyclo[2.2.2]oct-3-yl)(3-(3-fluorophenyl)benzamide),
(R)-N-(1-azobicyclo[2.2.2]oct-3-yl)(3-(3-methylphenyl)benzamide),
(R)-N-(1-azabicyclo[2.2.2]oct-3-yl)(3-(2-thienyl)benzamide),
(R)-N-(1-azabicyclo[2.2.2]oct-3-yl)(3-(3,5-dichlorophenyl)benzamide),
(R)-N-(1-azabicyclo[2.2.2]oct-3-yl)(3-(2-naphthyl)benzamide),
(R)-N-(1-azabicyclo[2.2.2]oct-3-yl)(3-(4-fluorophenyl)benzamide),
(R)-N-(1-azabicyclo[2.2.2]oct-3-yl)(5-(3-pyridyl)furan-2-carboxamide),
(R)-N-(1-azabicyclo[2.2.2]oct-3-yl)(5-(3-thienyl)furan-2-carboxamide),
(R)-N-(1-azabicyclo[2.2.2]oct-3-yl)(5-(2-benzo [b]furanyl)furan-2-carboxamide),
(R)-N-(1-azabicyclo[2.2.2]oct-3-yl)(5-(4-pyridyl)furan-2-carboxamide),
(R)-N-(1-azabicyclo[2.2.2]oct-3-yl)(5-(2-thienyl)furan-2-carboxamide),
(R)-N-(1-azabicyclo[2.2.2]oct-3-yl)(5-(3-methoxyphenyl)furan-2-carboxamide),
(R)-N-(1-azabicyclo[2.2.2]oct-3-yl)(5-(2-methoxyphenyl)furan-2-carboxamide),
(R)-N-(1-azabicyclo[2.2.2]oct-3-yl)(5-(4-fluorophenyl)furan-2-carboxamide),
(R)-N-(1-azabicyclo[2.2.2]oct-3-yl)(5-(2-naphthyl)furan-2-carboxamide),
(R)-N-(1-azabicyclo[2.2.2]oct-3-yl)(5-(3-methylphenyl)furan-2-carboxamide),
(R)-N-(1-azabicyclo[2.2.2]oct-3-yl)(5-(3-furyl)furan-2-carboxamide),
(R)-N-(1-azabicyclo[2.2.2]oct-3-yl)(5-(2-furyl)furan-2-carboxamide),
(R)-N-(1-azabicyclo[2.2.2]oct-3-yl)(5-(2-pyridyl)furan-2-carboxamide),
(R)-N-(1-azabicyclo[2.2.2]oct-3-yl)(5-(4-pyridyl)thiophene-2-carboxamide),
(R)-N-(1-azabicyclo[2.2.2]oct-3-yl)(5-(3-pyridyl)thiophene-2-carboxamide),
(R)-N-(1-azabicyclo[2.2.2]oct-3-yl)(5-(2-pyridyl)thiophene-2-carboxamide),
(R)-N-(1-azabicyclo[2.2.2]oct-3-yl)(4-(2-pyridyl)thiophene-2-carboxamide),
(R)-N-(1-azabicyclo[2.2.2]oct-3-yl)(4-(4-pyridyl)thiophene-2-carboxamide),
(R)-N-(1-azabicyclo[2.2.2]oct-3-yl)(4-(3-pyridyl)thiophene-2-carboxamide),
(R)-N-(1-azabicyclo[2.2.2]oct-3-yl)(5-(3-(N-acetylamino)phenyl)furan-2-carboxamide),
(R)-N-(1-azabicyclo[2.2.2]oct-3-yl)(5-(3-nitrophenyl)furan-2-carboxamide),
(R)-N-(1-azabicyclo[2.2.2]oct-3-yl)(5-(3-trifluoromethylphenyl)furan-2-carboxamide),
(R)-N-(1-azabicyclo[2.2.2]oct-3-yl)(5-(3-chlorophenyl)furan-2-carboxamide),
(R)-N-(1-azabicyclo[2.2.2]oct-3-yl)(5-(3-(N-acetylamino)phenyl)thiophene-2-carboxamide),
(R)-N-(1-azabicyclo[2.2.2]oct-3-yl)(5-(3-fluorophenyl)thiophene-2-carboxamide),
(R)-N-(1-azabicyclo[2.2.2]oct-3-yl)(5-(3-methoxyphenyl)thiophene-2-carboxamide),
(R)-N-(1-azabicyclo[2.2.2]oct-3-yl)(5-(3-ethoxyphenyl)thiophene-2-carboxamide),
(R)-N-(1-azabicyclo[2.2.2]oct-3-yl)(5-(3,5-dimethylisoxazol-4-yl)furan-2-carboxamide),
(R)-N-(1-azabicyclo[2.2.2]oct-3-yl)(5-(3,5-dimethylisoxazol-4-yl)thiophene-2-carboxamide),
(R)-N-(1-azabicyclo[2.2.2]oct-3-yl)(5-(3-aminophenyl)thiophene-2-carboxamide),
(R)-N-(1-azabicyclo[2.2.2]oct-3-yl)(5-(3-pyridyl)thiophene-3-carboxamide),
(R)-N-(1-azabicyclo[2.2.2]oct-3-yl)[5-(4-chlorophenyl)furan-2-carboxamide),
(R)-N-(1-azabicyclo[2.2.2]oct-3-yl)(5-(3-pyridyl)thiazole-3-carboxamide),
(R)-N-(1-azabicyclo[2.2.2]oct-3-yl)(5-(4-pyridyl)thiazole-3-carboxamide),
(R)-N-(1-azabicyclo[2.2.2]oct-3-yl)(5-(3-(N,N-dimethylamino)phenyl)thiophene-2-carboxamide),
(R)-N-(1-azabicyclo[2.2.2]oct-3-yl)(5-(8-quinolinyl)thiophene-2-carboxamide),
(S)-N-(1-azabicyclo[2.2.2]oct-3-yl)(5-(3-pyridyl)thiophene-2-carboxamide);
(S)-N-(1-azabicyclo[2.2.2]oct-3-yl)(5-(4-pyridyl)thiophene-2-carboxamide),
(S)-N-(1-azabicyclo[2.2.2]oct-3-yl)(5-(2-pyridyl)thiophene-2-carboxamide),
(S)-N-(1-azabicyclo[2.2.2]oct-3-yl)(5-phenylthiophene-2-carboxamide),
(R)-N-(1-azabicyclo[2.2.2]oct-3-yl)(5-phenylthiophene-3-carboxamide),
(R)-N-(1-azabicyclo[2.2.2]oct-3-yl)(4-phenylthiophene-2-carboxamide),
(R)-N-(1-azabicyclo[2.2.2]oct-3-yl)(5-(3-cyanophenyl)thiophene-2-carboxamide),
(R)-N-(1-azabicyclo[2.2.2]oct-3-yl)(5-(3-(N-methylamino)phenyl)thiophene-2-carboxamide),
(R)-N-(1-aza-bicyclo[2.2.2]oct-3-yl)(5-(3-hydroxyphenyl)thiophene-2-carboxamide),
(R)-N-(1-azabicyclo[2.2.2]oct-3-yl)(5-(3-pyridylamino)thiophene-2-carboxamide),
(R)-N-(1-azabicyclo[2.2.2]oct-3-yl)(5-(3-chlorophenyl)thiophene-2-carboxamide),
(R)-N-(1-azabicyclo[2.2.2]oct-3-yl)(5-(3-(4-morpholinyl)phenyl)thiophene-2-carboxamide),
(R)-N-(1-azabicyclo[2.2.2]oct-3-yl)(5-(3-(aminomethyl)phenyl)thiophene-2-carboxamide),
(R)-N-(1-azabicyclo[2.2.2]oct-3-yl)(5-phenoxythiophene-2-carboxamide),
(R)-N-(1-azabicyclo[2.2.2]oct-3-yl)(5-(3-aminophenyl)furan-2-carboxamide),
(R)-N-(1-azabicyclo[2.2.2]oct-3-yl)(5-(3-(N,N-dimethylamino)phenyl)furan-2-carboxamide),
(R)-N-(1-azabicyclo[2.2.2]oct-3-yl)(5-(3-formylphenyl)thiophene-2-carboxamide), or
(R)-N-(1-azabicyclo[2.2.2]oct-3-yl)(5-(3-(hydroxymethyl)phenyl)thiophene-2-carboxamide), or a pharmaceutically-acceptable salt thereof.

15. A combination according to claim 13, wherein said statin is rosuvastatin or a pharmaceutically-acceptable salt thereof and
said α7-nAChR agonist is selected from:
spiro[1-azabicyclo[2.2.2]octane-3,5′-oxazolidine]-2′-one;
N-(1-Azabicyclo[2.2.2]oct-3-yl)[E-3-(2-thienyl)propenamide], or
(2′R)-5′-(3-furanyl)spiro[1-azabicyclo[2.2.2]octane-3,2′-(3′H)-furo[2,3-b]pyridine] or a pharmaceutically-acceptable salt thereof.

16. A pharmaceutical composition comprising a combination according to claim 13 together with a pharmaceutically acceptable diluent or carrier.

17. A method providing neuroprotection or analgesia for the treatment or prophylaxis of a condition or disorder involving reduced cholinergic function selected from Alzheimer's disease, cognitive or attention disorders, anxiety, depression, smoking cessation, schizophrenia, Tourette's syndrome, and Parkinson's disease which method comprises administering to a patient suffering therefrom a therapeutically effective amount of a pharmaceutical composition as defined in claim 16.

17. The method according to claim 16, for treatment or prophylaxis of Alzheimer's disease.

18. A method providing neuroprotection or analgesia for treatment or prophylaxis of a condition or disorder involving reduced cholinergic function selected from Alzheimer's disease, cognitive or attention disorders, anxiety, depression, smoking cessation, schizophrenia, Tourette's syndrome, and Parkinson's disease which method comprises administering to a patient suffering therefrom a therapeutically effective amount of a combination as defined in claim 13.

19. A method for the treating a condition or disorder involving reduced cholinergic function selected from Alzheimer's disease, cognitive or attention disorders, anxiety, depression, smoking cessation, schizophrenia, Tourette's syndrome, and Parkinson's disease which method comprises administering to a patient suffering therefrom a therapeutically effective amount of a a statin selected from atorvastatin, cerivastatin, fluvastatin, lovastatin, pravastatin sodium, simvastatin or rosuvastatin, or a pharmaceutically-acceptable salt thereof and
an α7-nAChR agonist.

20. The method according to claim 19, for treatment or prophylaxis of Alzheimer's disease.

21. The method according to claim 19, wherein said statin is rosuvastatin or a pharmaceutically-acceptable salt thereof and said α7-nAChR agonist is selected from:
spiro[1-azabicyclo[2.2.2]octane-3,5′-oxazolidine]-2′-one;
N-(1-Azabicyclo[2.2.2]oct-3-yl)[E-3-(2-thienyl)propenamide], or
(2′R)-5′-(3-furanyl)spiro[1-azabicyclo[2.2.2]octane-3,2′-(3′H)-furo[2,3-b]pyridine] or a pharmaceutically-acceptable salt thereof.
